(19) United States
(12) Patent Application Publication
(10) Pub. No.: US 2008/0262740 A1
(43) Pub. Date: Oct. 23, 2008

(54) PERMITTIVITY-BASED MATERIAL SENSOR

(75) Inventor: Michael D. Potter, Churchville, NY (US)

Correspondence Address: JAECKLE FLEISCHMANN & MUGEL, LLP, 190 Linden Oaks, ROCHESTER, NY, 14625-2812, US

(73) Assignee: Infotonics Technology Center, Inc.

(21) Appl. No.: 11/738,214

(22) Filed: Apr. 20, 2007

(51) Int. Cl. G01N 33/48; G01N 15/00; G01N 31/00

(52) U.S. Cl. 702/19; 702/23; 73/53.01

(57) ABSTRACT

A system for sensing a presence and/or a concentration of a target substance in a fluid has a sensor and a processor coupled to the sensor. The sensor has a test probe having at least first and second test electrodes, wherein at least the first test electrode is functionalized to create a permittivity change in the area between the first and second test electrodes in the presence of the target substance. The sensor also has a reference probe having at least first and second reference electrodes. The processor is configured to determine at least one permittivity-based metric for the test probe; determine the at least one permittivity-based metric for the reference probe; and determine the presence and/or the concentration of the target substance based on the at least one permittivity-based metric for the test probe and the at least one permittivity-based metric for the reference probe. Related methods are disclosed.

### FIELD

The claimed invention generally relates to fluid component measuring systems and, more particularly, to a system and method for detecting and/or measuring a concentration of at least one substance within a fluid.

### BACKGROUND

Advances in society's medical knowledge have led to greater awareness and need for testing and characterization of bodily fluids. These fluids, such as blood, contain useful information which, if known, can assist medical professionals and patients to improve the patient's health, maintain a patient's health, monitor for health changes and/or possible drug interactions, and in general have a more complete picture of a patient's medical condition. For example, blood samples are frequently drawn from patients in order to measure glucose and cholesterol levels. Knowledge of glucose levels can be critical to helping patients administer proper doses of insulin when dealing with diabetes. As another example, cholesterol levels are important to track when considering the health of a patient's heart and circulatory system.

According to the American Diabetes Association, there are over twenty million people in the United States who have diabetes. The US Department of Health and Human Services recommends that people over the age of twenty should have their cholesterol checked every five years. With over two hundred million adults in the Unites States alone, that would imply that over forty million adults should have their cholesterol tested every year. Just looking at the need for glucose and cholesterol testing, there is a staggering need for solutions which can meet these testing needs. Unfortunately, not everyone who needs testing has the opportunity to be tested, since test methods can be expensive. Furthermore, even those who can afford current testing methods often avoid testing because the tests may be inconvenient. For example, whether testing for glucose, cholesterol, or any number of other target substances, patients are often required to take the time to visit a healthcare facility, wait for their turn to meet with a medical professional, have their blood drawn, wait for the sample to go to a lab for testing, and finally be contacted by a medical professional when the results are ready. Such delays reduce patient compliance with recommended testing frequency. The hassle of the testing also limits the number of times physicians are willing to ask for samples, knowing the inconvenience to the patient, despite the fact that more frequent data can often be helpful when treating or diagnosing a condition. As a result, there is a real need for lower-cost, reliable, and more convenient fluid testing.

In response to this need, a variety of products for testing bodily fluids has been developed to allow people to extract fluids at home and test them for various substances. For example, there are numerous glucose testing devices which prick the skin, draw an amount of blood, and through a variety of testing mechanisms measure the glucose level in the blood. Because such tests require the drawing of fluid, they are necessarily invasive and therefore can be uncomfortable since the mechanism used to draw blood must enter the skin far enough to reach the capillaries which lie among nerve tissue. As an alternative, some non-invasive testing equipment has been developed, such as optical devices which examine the spectral response of the vitreous humor within the eye. Unfortunately, such devices can be bulky and require a high level of expertise to administer.

Therefore, there is a need for a less expensive, less invasive, more reliable, and more convenient system and method for testing fluids, in particular bodily fluids, for a variety of substances.

### SUMMARY

A sensor is disclosed. The sensor has a test probe having at least a first test electrode and a second test electrode, wherein at least the first test electrode is functionalized to create a permittivity change in the area between the first and second test electrodes in the presence of a target substance. The sensor also has a reference probe having at least a first reference electrode and a second reference electrode. The sensor further has a substrate which supports the test probe and the reference probe, and which may be configured to be coupled to a processor for operation of the test probe and the reference probe.

A system for sensing a concentration of a target substance in a fluid is also disclosed. The system has a sensor and a processor coupled to the sensor. The sensor has a test probe having at least a first test electrode and a second test electrode, wherein at least the first test electrode is functionalized to create a permittivity change in the area between the first and second test electrodes in the presence of the target substance. The sensor also has a reference probe having at least a first reference electrode and a second reference electrode. The processor is configured to determine at least one permittivity-based metric for the test probe. The processor is also configured to determine the at least one permittivity-based metric for the reference probe. The processor is further configured to determine a concentration of the target substance based on the at least one permittivity-based metric for the test probe and the at least one permittivity-based metric for the reference probe.

A method of determining a concentration of a target substance in a fluid is disclosed. A test probe having a first test electrode and a second test electrode is contacted with the fluid. At least the first test electrode is functionalized to create a permittivity change in the area between the first and second test electrodes in the presence of the target substance. A reference probe having a first reference electrode and a second reference electrode is contacted with the fluid. A permittivity-based metric is determined for the test probe between the first test electrode and the second test electrode. A permittivity-based metric is determined for the reference probe between the first reference electrode and the second reference electrode. The presence of the target substance and/or the concentration of the target substance is determined based on the test probe permittivity-based metric and the reference probe permittivity-based metric.

### BRIEF DESCRIPTION OF THE DRAWINGS

FIG. 1 schematically illustrates one embodiment of a sensor for use in detecting and/or determining a concentration of target substance in a fluid.

FIGS. 2A and 2B schematically illustrate an embodiment of functionalized electrodes entering a fluid and reacting with a target substance in the fluid, respectively.

FIG. 3 schematically illustrates an embodiment of a comparison of capacitance between a reference probe and a functionalized test probe before and after insertion into a fluid.

FIGS. 4A-4C schematically illustrate embodiments of permittivity-based material sensors.

FIGS. 5A-5F schematically illustrate further embodiments of sensors for use in detecting and/or determining a concentration of a target substance in a fluid.

FIG. 6 schematically illustrates an embodiment of a system for detecting a target substance and/or sensing a concentration of a target substance in a fluid.

FIGS. 7A and 7B schematically illustrate an embodiment of a sensor being actuated through a subject's epidermis and into contact with bodily fluid.

FIG. 8 schematically illustrates one embodiment of a sensor array having a plurality of sensors for detecting and/or determining a concentration of one or more target substances in a fluid.

FIG. 9 schematically illustrates an embodiment of a method for determining a concentration of a target substance in a fluid.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

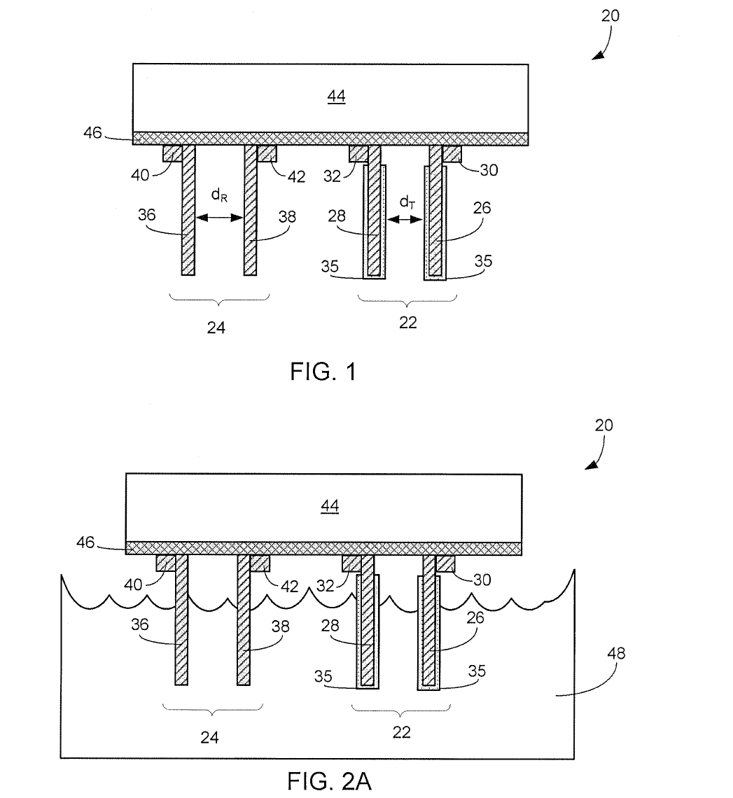

FIG. 1 schematically illustrates a sensor 20 which can be used to detect he presence of and/or determine a concentration of a target substance in a fluid and which is able to ignore interference from non-target substances by comparing results to a real-time reference. The sensor 20 has a test probe 22 and a reference probe 24. In this embodiment, the test probe 22 has a first test electrode 26 and a second test electrode 28 separated by a known distance d_{T}. The first and second test electrodes 26, 28 are conductive, and each can be electrically coupled to a respective contact 30, 32 which may be provided as coupling points for other circuitry (not shown) which can operate the test probe 22. Such other circuitry need not be part of the sensor 20. The first and second test electrodes 26, 28 can be bars, wires, rods, plates, or even microneedles. The first and second test electrodes 26, 28 may be constructed from a variety of conductive materials, such as aluminum, copper, zinc, palladium, silver, gold, titanium, and other metals, alloys, or any combination thereof. The first and second test electrodes are functionalized to create a permittivity change in the area between the first and second test electrodes 26, 28 in the presence of a target substance.

A functionalized electrode is one which has been treated or coated with a material 35 that will either cause hybridization or a chemical bond to occur in the presence of the target substance. Those skilled in the art may choose or design a variety of materials to functionalize an electrode, depending on the target substance. For example, if a biological material is a target, such as a specific protein, bacteria, spore, mycroplasma, prion, or virus, a complimentary oligonucleotide may be synthesized or chosen which will hybridize or bind to the target substance. If a chemical is the target, then the functionalized electrode may have a chemical or material coating designed to react or bond with the target substance.

In the embodiment of FIG. 1, the reference probe 24 has a first reference electrode 36 and a second reference electrode 38 separated by a known distance d_{R}. The first and second reference electrodes 36, 38 are conductive, and each may be electrically coupled to a respective contact 40, 42 which may be provided as a coupling point for other circuitry (not shown) which can operate the reference probe 24. Such other circuitry need not be part of the sensor 20. The first and second reference electrodes 36, 38 can be bars, wires, rods, plates, or even microneedles. The first and second reference electrodes 36, 38 may be constructed from a variety of conductive materials, such as aluminum, copper, zinc, palladium, silver, gold, titanium, and other metals, alloys, or any combination thereof.

The sensor 20 also has a substrate 44 which supports the test probe and the reference probe. The substrate may be made from a variety of materials, such as, but not limited to, silicon, glass, polymer, and quartz. If the substrate 44 is conductive, or even semiconductive, it may be desirable to have an insulating layer on the substrate 44 between the substrate and the electrodes 26, 28, 36, and 38. In the embodiment of FIG. 1, the substrate 44 is silicon, and an insulating layer 46 has been provided, such as, for example, silicon-dioxide.

As mentioned above, the first test electrode 26 and the second test electrode 28 are functionalized to create a permittivity change in the area between the first and second test electrodes 26, 28 in the presence of a target substance. The electrodes 26, 28, 36, 38 of the sensor 20 may be brought into contact with a fluid under test. If the fluid contains the target substance, then the target substance can be linked or bonded to the test electrodes 26, 28 due to the functionalization. Since the reference electrodes 36, 38 are not functionalized the target substance will not be linked or bonded to the reference electrodes 36, 38.

Any hybridization or reaction which occurs with the functionalized electrodes 26, 28 adds substance between the test probe electrodes 26, 28. This added material has a relative permittivity that is different than the relative permittivity of the overall fluid being tested, and therefore also different from the un-functionalized reference probe 24. Therefore, the permittivity of the area between the test probe electrodes 26, 28 can differ from the permittivity of the area between the reference probe electrode 36, 38 depending on the concentration of a target substance in a fluid being tested by the sensor 20. While permittivity and any corresponding change in permittivity may be determined for the area between a pair of electrodes, there are other permittivity-based metrics which may be measured as well, such as capacitance, impedance, and dielectric constant. As permittivity changes, so do each of these metrics. For convenience, further discussion of the embodiments will use capacitance measurement as the permittivity-based metric which is the sensor 20 can be used to monitor. It will be understood by those skilled in the art, however, that impedance or dielectric constant could also be used in place of capacitance since all are related to permittivity. Similarly, for convenience, the examples will focus on hybridization occurring with the functionalized electrodes, although it should be understood that other embodiments could alternatively have functionalized electrodes which are designed to react with a targeted chemical substance rather than have a hybridization.

In the example of FIG. 1, therefore, the test probe 22 is functionalized such that hybridization occurs in the presence of a specific target substance. The hybridization adds a substance between the test probe electrodes 26, 28. This added material has a relative permittivity that is different than the relative permittivity of the overall fluid being tested and therefore also different from the relative permittivity of the fluid between the reference electrodes 36, 38. Therefore, the capacitance of the test probe 22 and the capacitance of the reference probe 24 can differ in the presence of a targeted substance. Upon identification that a difference in capacitance exists between the test probe 22 and the reference probe 24, a Boolean-type determination can be made that the target substance is present in the fluid. In other embodiments, the sensor 20 may be used to monitor the rate of change of the capacitance. The rate of change of the capacitance (or other permittivity-based metric) can be related to the rate of hybridization which is based on the concentration of the target substance. Therefore, a device coupled to the sensor 20 and monitoring the rate of change of the capacitance as well as the final difference between the steady state capacitance of the test probe 22 and the reference probe 24 can determine an effective concentration or level of the target substance.

As a non-limiting example, consider an embodiment where the electrodes 26, 28, 36, and 38 are 100 μm long and 10 μm wide with a separation of 1 μm. The thickness of the individual electrodes does not affect the electrical analysis, and therefore, the thickness of the electrodes may be selected based on a variety of factors, including balancing the often competing needs of strength (possibly thicker electrodes needed) and real estate (possibly thinner electrodes needed). The capacitance of the reference probe 24 is:

$${C}_{ref} = \frac{{ɛ}_{0} {ɛ}_{i} A}{{d}_{R}}$$

where: C_{ref} is the capacitance of the reference probe;
ε₀ is the permittivity of free space;
εᵢ is relative permittivity (dielectric constant) of the liquid being analyzed;
A is the area of one of the reference electrodes; and
d_{R} is the distance between the two reference electrodes 36, 38.

The capacitance of the test probe 22 is:

$${( Ctest )}^{- 1} = \frac{{d}_{1}}{{ɛ}_{0} {ɛ}_{iT} A} + \frac{{d}_{2}}{{ɛ}_{0} {ɛ}_{i} A} + \frac{{d}_{3}}{{ɛ}_{0} {ɛ}_{iF} A}$$

where: Cₜₑₛₜ is the capacitance of the test probe;
ε₀ is the permittivity of free space;
ε_{iT} is the relative permittivity (dielectric constant) of the target substance;
d₁ is the thickness of the target substance which has been hybridized between the test electrodes;
εᵢ is relative permittivity (dielectric constant) of the liquid being analyzed;
d₂ is the thickness of the liquid being analyzed between the test electrodes;
ε_{iF} is relative permittivity (dielectric constant) of the functionalization 35;
d₃ is the thickness of the functionalization between the test electrodes; and
A is the area of one of the test electrodes 26, 28.

Capacitance may be measured in a variety of ways familiar to those skilled in the art.

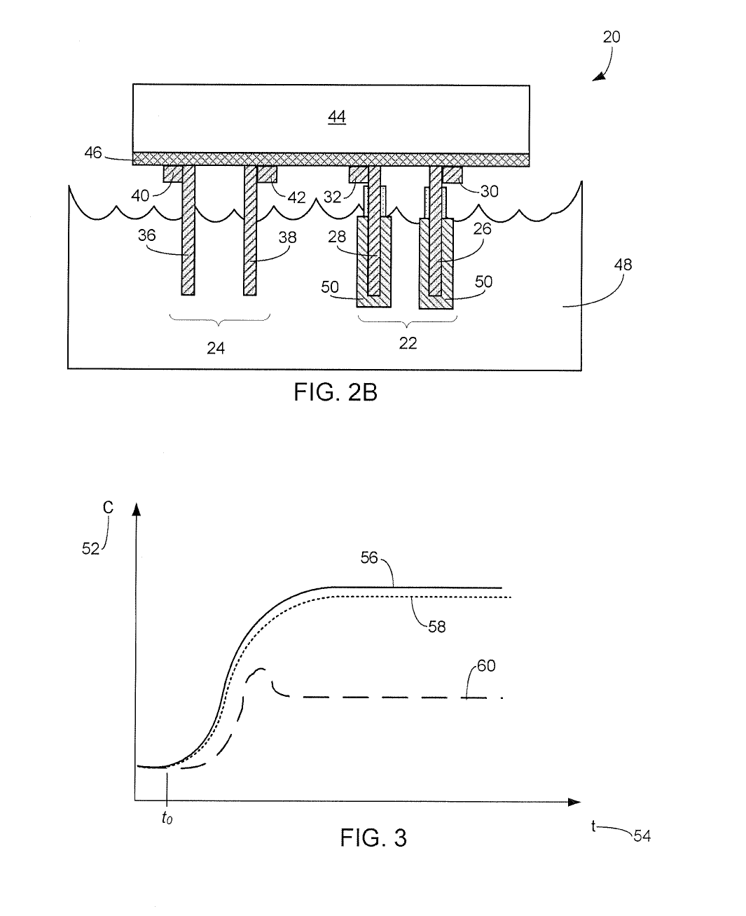

FIGS. 2A and 2B schematically illustrate an embodiment of functionalized electrodes 26, 28 entering a fluid 48 and hybridizing (in the case of a biological target substance) or reacting (in the case of a chemical target substance) with a target substance in the fluid, respectively. The fluid 48 is illustrated generically, since the sensor 20 can be used for testing of a variety of fluids from laboratory fluids, to in vivo body fluids, to agricultural fluids, dairy fluids, industrial fluids, and food industry fluids. FIG. 2A schematically illustrates the reference probe 24 and the test probe 22 initially entering the fluid 48 at a start time of t₀. Assuming the targeted substance is present in the fluid, hybridization of the targeted substance begins to occur on the test electrodes 26, 28.

For the sake of an example only, assume that the liquid 48 is de-ionized water. In general, however, the actual characteristics of the intervening fluid do not matter as long as it is not a pure conductor. The sensor 20 could be used to determine the presence of and/or the concentration of a target substance in a variety of fluids. For this example only, assume the actual physical separation between the reference electrodes 36, 38 is 1 μm (which is 10,000 angstroms) and assume the actual physical separation between the functionalized test electrodes 26, 28 is also 1 μm. The relative permittivity of the de-ionized water is approximately 84. Therefore, after initial insertion of the test probe 22 and the reference probe 24 (FIG. 2A), before any hybridization, the free space equivalent distance between the test electrodes 26, 28 is 119 angstroms (=10,000/84). Similarly, the free space equivalent distance between the reference electrodes 36, 28 is also 119 angstroms. The functionalization capacitance is generally small enough to ignore. Therefore, this example does not take the functionalization capacitance into consideration in this example.

For this continued example only, assume that the test probe 22 is functionalized for glucose hybridization. Referring to FIG. 2B, at some time after to, a thickness of hybridized glucose 50 will form on each of the functionalized test electrodes 26, 28. For this example only, assume that the hybridized glucose 50 has a thickness of 50 angstroms on all sides of the functionalized test electrodes 26, 28. The 50 angstroms of hybridized glucose on the outside of the electrodes (i.e. not between the electrodes) has no effect. The hybridized glucose 50 on the inner portion of the electrodes 26, 28 has a total thickness of 100 angstroms. Knowing that the relative permittivity of glucose is approximately 3, we can calculate the free space equivalent distance between the test electrodes 26, 28.

The free space equivalent distance between the electrodes 26, 28 of the functionalized test probe 22 is the thickness of the intervening fluid (10,000 angstroms-100 angstroms) divided by the relative permittivity of the intervening fluid (84) plus the thickness of the intervening glucose (100 angstroms) divided by the relative permittivity of the intervening glucose (3). Therefore, in this example, the free space equivalent distance between the test electrodes 26, 28 after hybridization is 151 angstroms. Recall in this example that the free space equivalent between the reference electrodes 36, 28 is 119 angstroms. Capacitance varies inversely proportional to the distance between the electrodes. Since, in this example, the free space equivalent spacing between the test probe electrodes 26, 28 has increased by more than 25%, the difference in capacitance between the reference probe 24 and the functionalized test probe 22 is significant. This difference can be used to make a determination that the target substance (in this example, glucose) is present. Additionally, in some embodiments, the rate of change of a permittivity-based metric, such as capacitance, impedance, or dielectric constant can be monitored to determine a concentration level of a target substance.

FIG. 3 schematically illustrates an embodiment of a comparison of capacitance between a reference probe and a functionalized test probe before and after insertion into a fluid. Capacitance 52 is plotted as a function of time 54. The capacitance of the reference probe 56 is shown versus time 54. An expected test probe capacitance for no hybridization 58 is shown versus time 54. Finally, the functionalized test probe capacitance with a hybridized target 60 is shown versus time 54. The reference probe and the test probe were brought into contact with the fluid under test at time to.

Curves such as those in FIG. 3, where permittivity-based metrics are measured over time, may be collected for various known concentrations of a target substance. By storing the information in such curves, by storing a mathematical relationship which characterizes all or part of such curves, or by storing a look-up table based on information from such curves, a sensor can be calibrated to be able to detect the presence and/or the concentration of a particular target substance. The measurement of the reference probe works to reduce measurement errors in the test probe by reacting to contaminants such as various ions in the liquid under test in a similar fashion to the test probe. By characterizing the capacitance of the test electrode relative to the reference electrode versus time, the measurements possible by the sensor can have increased accuracy and reliability.

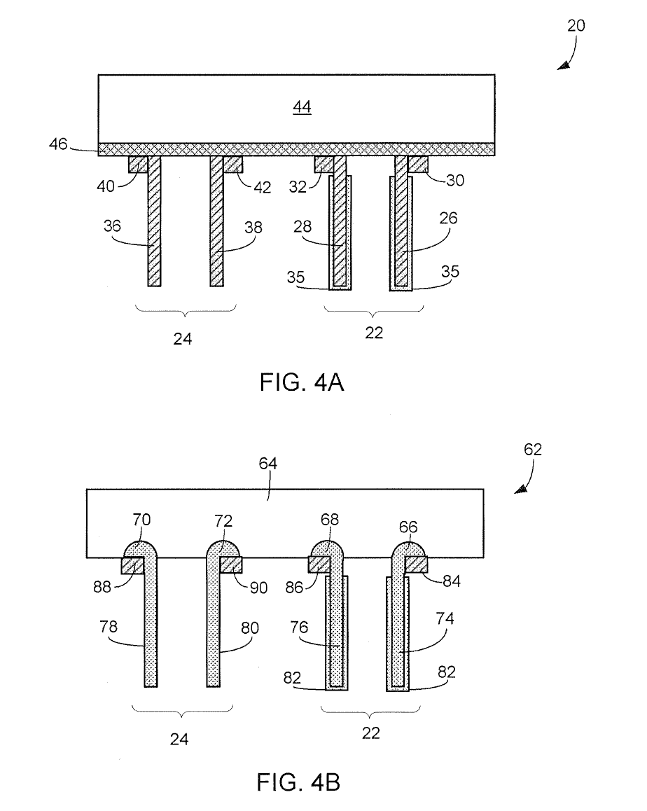

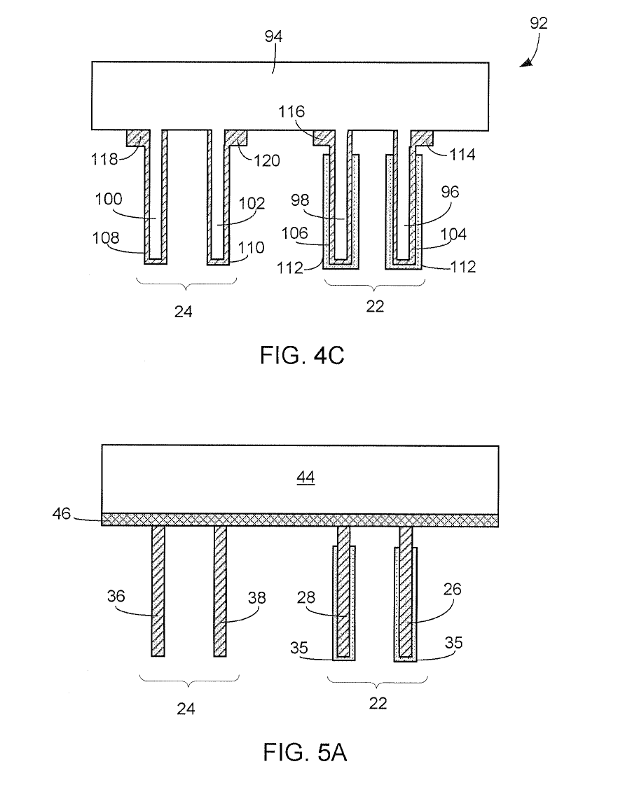

FIGS. 4A-4C schematically illustrate embodiments of permittivity-based material sensors. The sensor 20 embodied in FIG. 4A has been discussed above. This sensor 20 has a substrate 44 which has been insulated on an electrode side, for example by oxidizing a silicon substrate 44 to form a silicon-dioxide insulating layer 46 on the substrate 44. Test electrodes 26, 28 and reference electrodes 36, 38 may be deposited, formed, or otherwise coupled onto the insulated substrate 44. The electrodes 26, 28, 36, 38 may be made from any desired metal, alloy, other conductor, or any combination thereof. A functionalization layer 35 has been deposited on both of the test electrodes 26, 28. The deposition of the functionalization layer 35 can be done by dipping the electrode in a liquid which then dries on the electrode, or the deposition can be done with a device similar to an ink-jet printhead by directly depositing a drop of functionalization liquid on the electrode so that it can dry. Sensor configurations, such as the embodiment of FIG. 4A are relatively simple to manufacture using either standard manufacturing techniques, or even MEMS micro-fabrication techniques. Contact pads 30, 32, 40, and 42 may be provided and each coupled to an electrode 26, 28, 36, 38 in order to provide a place where the sensor 20 can be coupled to external circuitry.

FIG. 4B schematically illustrates an alternate embodiment of a sensor 62 which is manufactured a different way, but which may be operated similarly to the previously described embodiments. The sensor 62 has a substrate 64 which has been doped to create separate semiconductor areas 66, 68, 70, and 72. The semiconductor areas 66, 68, 70, and 72 have been etched to form test electrodes 74 and 76 and reference electrodes 78 and 80, respectively. A functionalization layer 82 can be formed on the test electrodes 74, 76. Additionally, contact pads 84, 86, 88, and 90 may be provided in contact with respective doped regions 66, 68, 70, and 72 to provide a place where the sensor 62 can be coupled to external circuitry.

FIG. 4C schematically illustrates an alternate embodiment of a sensor 92 which is manufactured in yet a different way, but which may be operated similarly to the previously described embodiments. The sensor 92 has a substrate 94 which has been etched to form test electrode supports 96, 98 and reference electrode supports 100, 102. The substrate 94 in this embodiment is non-conductive, or has been insulated (for example by oxidation) to make it non-conductive. Test electrodes 104, 106, and reference electrodes 108, and 110 are deposited respectively onto electrode supports 96, 98, 100, and 102. A functionalization layer 112 can be formed on test electrodes 104, 106. Additionally, contact pads 114, 116, 118, and 120 may be formed to provide a place where the sensor 92 can be coupled to external circuitry.

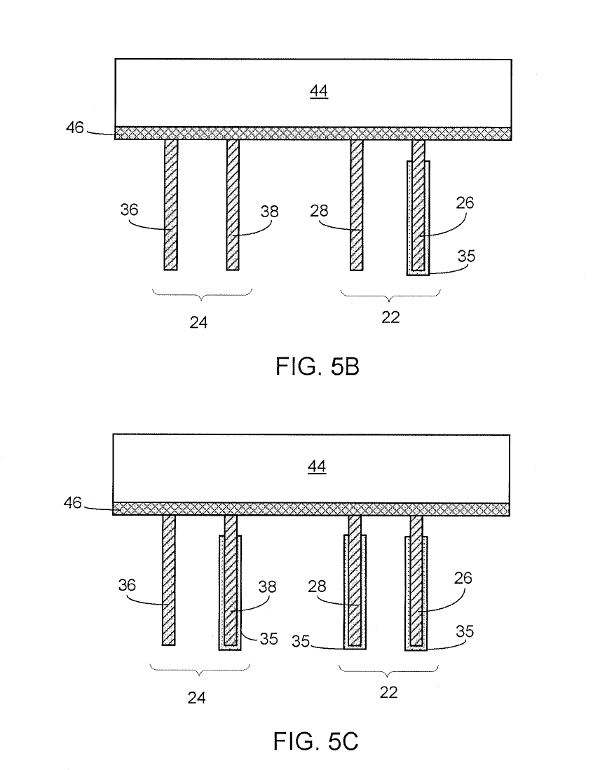

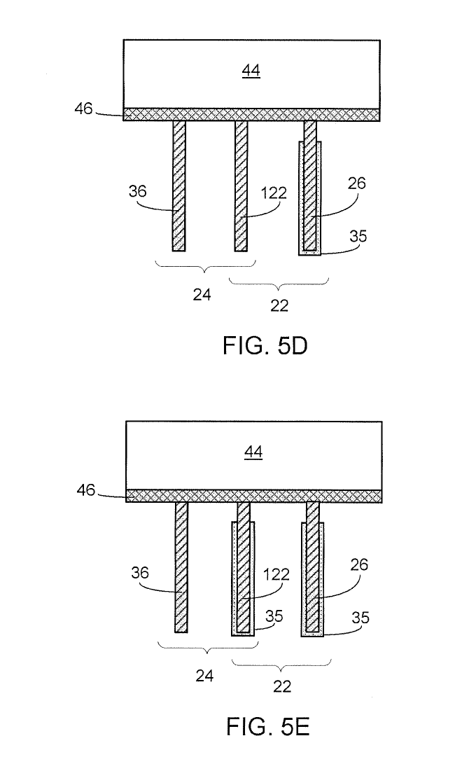

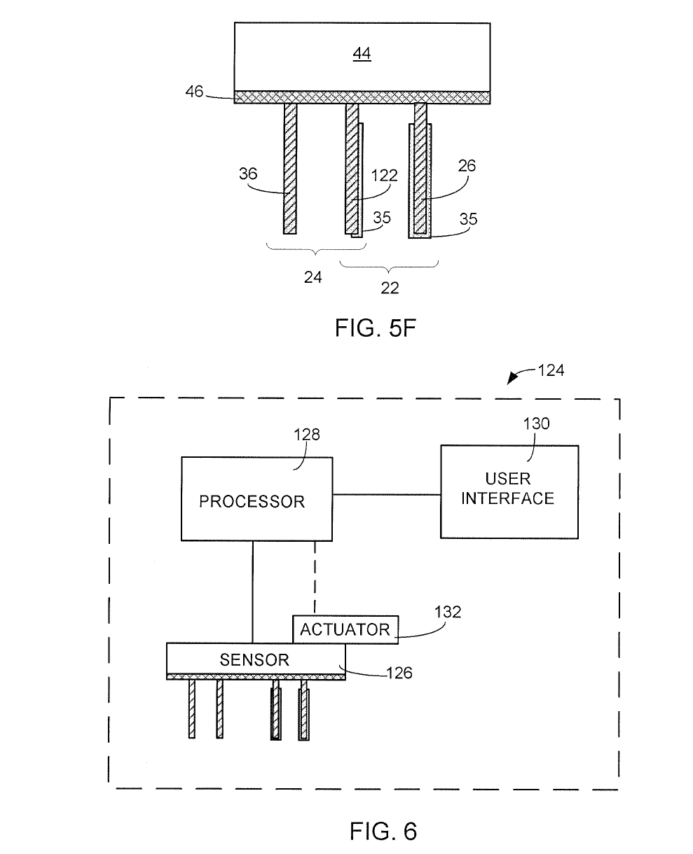

FIGS. 5A-5F schematically illustrate further embodiments of permittivity-based material sensors. The embodiment of FIG. 5A is similar to the sensors embodied in FIGS. 1 and 4A, although optional contact pads are not shown for simplicity. A substrate 44 and an insulator 46 support one pair of test electrodes 26, 28 which make up a test probe 22. The substrate 44 and the insulator 46 also support one pair of reference electrodes 36, 38 which make up reference probe 24. In the embodiment of FIG. 5A, both of the test electrodes 26, 28 are functionalized 35 to create a permittivity change in the area between the first and second test electrodes 26, 28 in the presence of a target substance. The operation of this type of device has been discussed above.

The sensor embodied in FIG. 5B is similar to the sensor of FIG. 5A, with the exception that only one of the test electrodes is functionalized. A substrate 44 and an insulator 46 support one pair of reference electrodes 36, 38 which make up reference probe 24. The substrate 44 and the insulator 46 also support one pair of test electrodes 26, 28 which make up test probe 22. In the embodiment of FIG. 5B, however, only one of the test electrodes 26 is functionalized 35 to create a permittivity change in the area between the first and second test electrodes 26, 28 in the presence of a target substance. This type of sensor will operate similarly to the embodiments described above, however, since only one test electrode is functionalized, the change in a permittivity-based metric for the area between the first and second test electrodes 26, 28 will not be as large for a given concentration of target substance.

The sensor embodied in FIG. 5C is similar to the sensor of FIG. 5A, with the exception that one of the reference electrodes is also functionalized. A substrate 44 and an insulator 46 support one pair of reference electrodes 36, 38 which make up reference probe 24. The substrate 44 and the insulator 46 also support one pair of test electrodes 26, 28 which make up test probe 22. Both of the test electrodes 26, 28 have been functionalized 35 to create a permittivity change in the area between the first and second test electrodes 26, 28 in the presence of a target substance. Additionally, in the embodiment of FIG. 5C, one of the reference electrodes 38 has been functionalized 35. While the single functionalized reference electrode 38 will create a permittivity change in the area between the first and second reference electrodes, the non-functionalized reference probe 36 may still provide a reference permittivity-based metric for comparison to the test probe metric.

In the embodiments of FIGS. 5D-5F, the sensor only has three electrodes, rather than four. The three electrode sensor embodiments work by asking the center electrode to do double duty. For example, first a permittivity-based metric may be determined between a left electrode and the center electrode. Next, a permittivity-based metric may be determined between a right electrode and the center electrode. In the embodiment of FIG. 5D, a substrate 44 and an insulator 46 support a first test electrode 26, a first reference electrode 36, and a shared electrode 122. The test electrode 26 has been functionalized 35 to create permittivity change in the area between the first test electrode 26 and the shared electrode 122. This type of sensor will operate similarly to the embodiments described above, however, since only one test electrode is functionalized, the change in a permittivity-based metric for the area between the first test electrode 26 and the shared electrode 122 will not be as large for a given concentration of target substance.

In the embodiment of FIG. 5E, a substrate 44 and an insulator 46 support a first test electrode 26, a first reference electrode 36, and a shared electrode 122. The test electrode 26 and the shared electrode 122 have been functionalized 35 to create permittivity change in the area between the first test electrode 26 and the shared electrode 122. Since both the first test electrode 26 and the shared electrode 122 have been functionalized 35, the test probe 22 will operate similarly to the test probes described previously. While the shared functionalized reference electrode 122 will create a permittivity change in the area between the first reference electrode 36 and the shared electrode 122, the non-functionalized reference electrode 36 may still provide a reference permittivity-based metric for comparison to the test probe metric.

In the embodiment of FIG. 5F, a substrate 44 and an insulator 46 support a first test electrode 26, a first reference electrode 36, and a shared electrode 122. The test electrode 26 has been functionalized 35 for a target substance. The side of the shared electrode 122 facing the test electrode 26 has also been functionalized 35. Since both the first test electrode 26 and the side of the shared electrode 122 facing the first test electrode 26 have been functionalized, the test probe 22 will operate similarly to the test probes 22 described previously. Similarly, since both the first reference electrode 36 and the side of the shared electrode 122 facing the first reference electrode have not been functionalized, the reference probe 24, will operate similarly to the reference probes 24 described previously.

FIG. 6 schematically illustrates an embodiment of a system 124 for sensing the presence of a target substance in a fluid and/or a concentration of the target substance in the fluid. The system has a sensor 126 which can be any of the embodiments previously described or their equivalents. The system 124 also has a processor 128 coupled to the sensor 126 and configured to determine at least one test probe permittivity-based metric for the sensor 126. The processor 128 is also configured to determine at least one reference probe permittivity-based metric for the sensor 126. The processor 128 is further configured to determine the presence and/or the concentration of at least one target substance based on the test probe permittivity-based metric and the reference probe permittivity-based metric.

In some embodiments, the sensor 126 may be removeably coupled to the processor 128. The processor 128 should be construed broadly to include one or more microprocessors, computers, laptops, application specific integrated circuits (ASIC's), analog electronics, digital electronics, or any combination thereof. Furthermore, the processor 128 may be distributed, where some components are remotely coupled (for example over a network, over an internet, or over some type of wireless or optical connection) to the sensor 126. In other distributed environments, processor components involved in the analysis for the sensor may be fed data at a later time due to raw data or pre-processing data being stored in a memory until a connection is made with the distributed components.

In some embodiments, the system 124 will also have a user interface 130 coupled to the processor 128. The user interface 130 can provide a Boolean type “present/not-present” indication of whether a target substance is present in a fluid. In other embodiments, the interface 130 can alternately or additionally display a concentration of the target substance in the fluid. Other embodiments of a user interface 130 might show graphs of the permittivity-based metrics over time, such as capacitance, impedance, or dielectric constant.

In some embodiments, the sensor 126 may be coupled to an actuator 132. The actuator 132 may be manually activated, or the actuator 132 may be coupled to the processor 128 and activated by the processor 128. The actuator 132 may be configured to move the test probes on a sensor 126 from a retracted position where no subject fluid is in contact with the sensor 126 to an engaged position where the sensor 126 may contact a subject fluid which may have a target substance. The subject fluid may be tested in a variety of locations, from a laboratory environment, to a vessel, to within a pipe, or even in vivo, beneath a subject's skin.

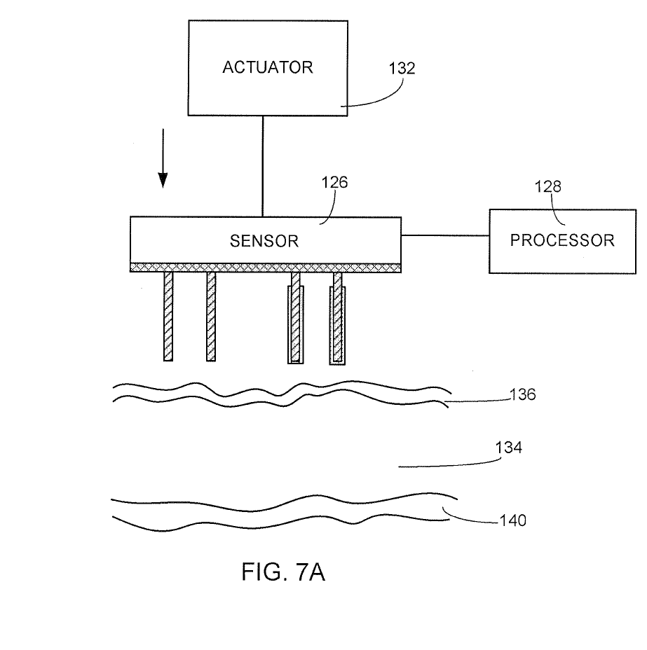

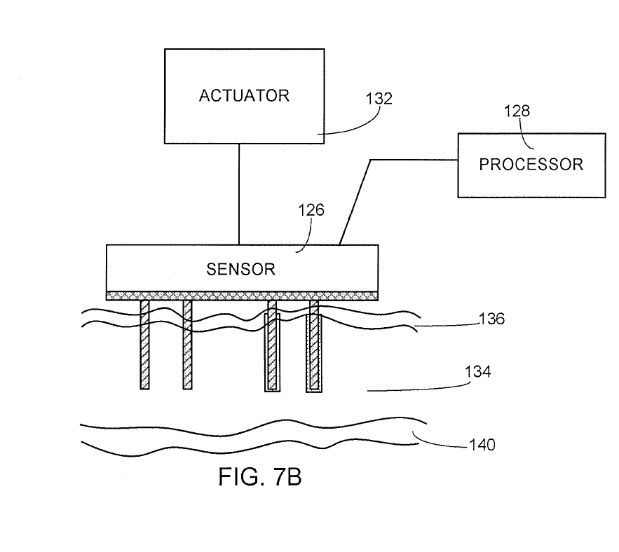

FIGS. 7A and 7B schematically illustrate an actuator 132 being used to actuate a sensor 126 from the retracted position in FIG. 7A to the engaged position of FIG. 7B. The subject fluid in this embodiment is the interstitial fluid 134 which is found below an epidermis layer 136 of the skin. The sensor electrodes may be sized to easily penetrate the epidermis 136 while not reaching the deeper layers of the skin 140 where capillaries and nerve tissue are situated. Measurements in such a system can occur as described in previous embodiments, while the sensor 126 is in vivo. Such a system can be fully or partially automated, is unobtrusive, and can be cost effective since only the sensor is disposable. These advantages reduce patient worry, apprehension, and discomfort, and help reduce costs.

Measurements with embodiments of the sensors and systems described herein, and their equivalents, are not limited to laboratory or in vivo testing. Such sensors and systems may be used in a variety of settings, such as agriculture, industry, food preparation, and even dairy fanning. The claimed sensors are suitable for inclusion in hand-held and/or portable sensing machines. The claimed sensors are also suitable for inclusion in pipes. As one example, dairy farmers often have a complex plumbing system which combines milk from numerous cows into pipes which deliver the milk to treatment or holding tanks. There are certain biological contaminants which it is desirable to test for in the milk. Generally, this testing is done on the milk in a storage tank. With a permittivity-based material sensor, however, sensors could be placed in the pipes from each cow, regularly monitoring each cow's output to see if the milk is up to desired health standards. Upon the discovery of tainted milk, the cow's milk line could be shut down, thereby reducing the chances of contaminating a large collection tank of milk.

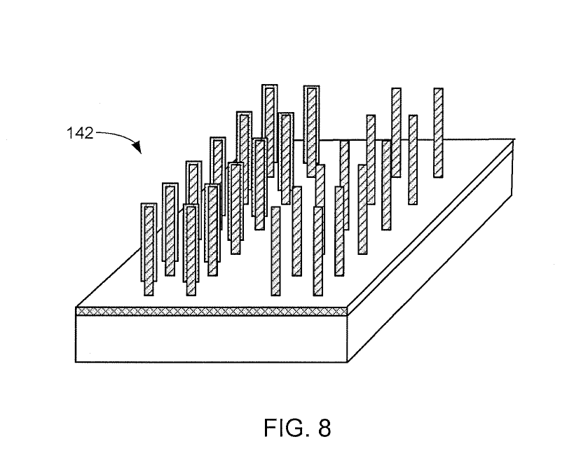

A variety of applications and uses for the sensors and systems described herein, and their equivalents will be apparent to those skilled in the art. It should additionally be noted that although one sensor was used in the example embodiments for simplicity in teaching the concepts, the sensor in the system could be a sensor array having a plurality of sensors, either in a one-dimensional array or a two-dimensional array. An embodiment of a sensor array 142 having a plurality of sensors is schematically illustrated in FIG. 8, the test probe portions of the one or more sensors being functionalized for one or more target substances. The advantage of a sensor array 142 is that it allows for redundant testing to increase confidence levels and/or testing of a variety of target substances all in one test. It should be apparent that other embodiments of sensor arrays 142 may have other configurations and combinations of sensors within the array 142. The example illustrated in FIG. 8 shows the sensor array having a plurality of sensors similar to the sensor discussed previously with regard to FIG. 5A. Other embodiments may use multiple sensors corresponding to the embodiments of FIGS. 1, 4A, 4B, 4C, 5A-5F, and their equivalents. Still other embodiments may also use any combination of sensors corresponding to the embodiments of FIGS. 1, 4A, 4B, 4C, 5A-5F, and their equivalents.

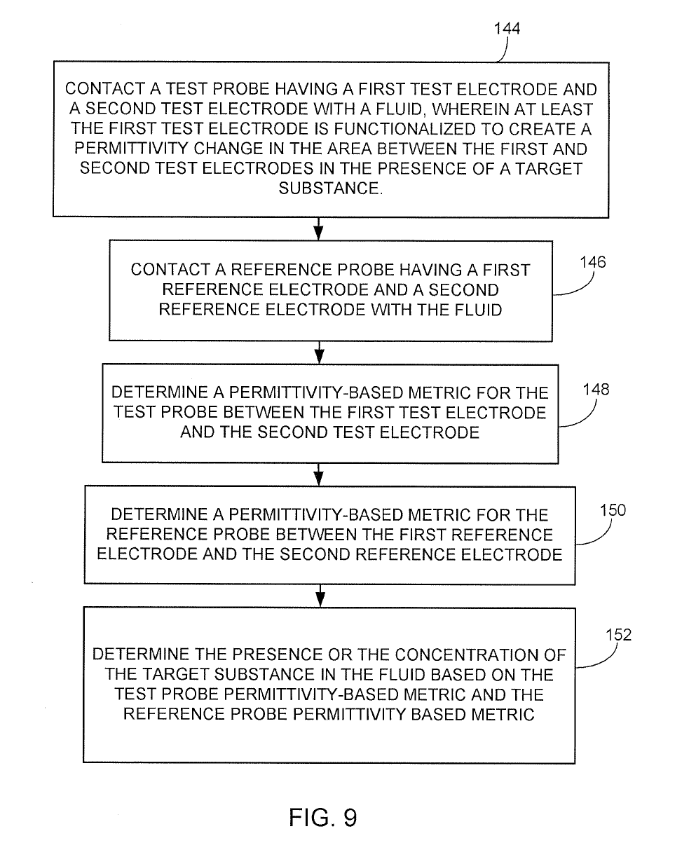

FIG. 9 illustrates one embodiment of a method of determining a concentration of a target substance in a fluid. A test probe having a first test electrode and a second test electrode is brought into contact 144 with the fluid, wherein at least the first test electrode is functionalized to create a permittivity change in the area between the first and second test electrodes in the presence of the target substance. A reference probe, having a first reference electrode and a second reference electrode is brought into contact 146 with the fluid. A permittivity-based metric is determined 148 for the test probe between the first test electrode and the second test electrode. A permittivity-based metric is also determined 150 for the reference probe between the first reference electrode and the second reference electrode. A concentration of the target substance is determined 152 based on the test probe permittivity-based metric and the reference probe permittivity-based metric. Depending on the embodiment, the determination of this target substance concentration can be as simple as a yes/no indication that the target substance is present, or it can involve providing an actual concentration based on calibration data.

Having thus described several embodiments of the claimed invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of the processing elements or sequences, or the use of numbers, letters, or other designations therefore, is not intended to limit the claimed processes to any order except as may be specified in the claims. Accordingly, the claimed invention is limited only by the following claims and equivalents thereto.

What is claimed is:

1. A sensor, comprising:
a test probe having at least a first test electrode and a second test electrode, wherein at least the first test electrode is functionalized to create a permittivity change in the area between the first and second test electrodes in the presence of a target substance;
a reference probe having at least a first reference electrode and a second reference electrode; and
a substrate which supports the test probe and the reference probe, and which is configured to be coupled to a processor for operation of the test probe and the reference probe.

2. The sensor of claim 1, wherein the second test electrode is also functionalized to create a permittivity change in the area between the first and second test electrodes in the presence of the target substance.

3. The sensor of claim 1, wherein the second test electrode and the second reference electrode comprise a shared electrode.

4. The sensor of claim 3, wherein the shared second test electrode and second reference electrode are functionalized such that no permittivity change occurs in the area between the first and second reference electrodes in the presence of the target substance.

5. The sensor of claim 2, wherein the second reference electrode is functionalized for the target substance.

6. The sensor of claim 1, wherein:
the first reference electrode comprises a first reference electrode support; and
the second reference electrode comprises a second reference electrode support.

7. The sensor of claim 1, wherein:
the first reference electrode comprises a first reference doped region; and
the second reference electrode comprises a second reference doped region.

8. The sensor of claim 1, wherein:
the first test electrode comprises a first test electrode support; and
the second test electrode comprises a second test electrode support.

9. The sensor of claim 1, wherein:
the first test electrode comprises a first test doped region; and
the second test electrode comprises a second test doped region.

10. The sensor of claim 1, wherein the first test electrode and the second test electrode are functionalized with a material selected from the group consisting of glucose oxidase and oligonucleotides.

11. The sensor of claim 1, wherein the target substance the first test electrode and the second test electrode are functionalized for is selected from the group consisting of glucose, cholesterol, potassium, a hormone, a vitamin, a biological agent, a bacteria, a virus, a spore, mycoplasma, a prion, and a protein.

12. The sensor of claim 1, wherein the substrate is selected from the group consisting of silicon, glass, polymer, and quartz.

13. The sensor of claim 1, wherein the substrate further comprises circuitry coupled to the reference probe and the test probe and configured to be coupled to a processor for operation of the test probe and the reference probe.

14. The sensor of claim 1, wherein the circuitry comprises CMOS technology.

15. The sensor of claim 1, wherein the second test electrode and the second reference electrode comprise a shared electrode.

16. The sensor of claim 1, wherein the substrate further comprises an insulator.

17. The sensor of claim 1 further comprising a plurality of test probes and at least one reference probe.

18. The sensor of claim 17, wherein the plurality of test probes and plurality of reference probes are arranged in a one-dimensional array.

19. The sensor of claim 17, wherein the plurality of test probes and plurality of reference probes are arranged in a two dimensional array.

19. A system for sensing a presence and/or a concentration of a target substance in a fluid, comprising:
a) a sensor, comprising:
i) a test probe having at least a first test electrode and a second test electrode, wherein at least the first test electrode is functionalized to create a permittivity change in the area between the first and second test electrodes in the presence of a target substance; and
ii) a reference probe having at least a first reference electrode and a second reference electrode; and
b) a processor coupled to the sensor and configured to:
i) determine at least one permittivity-based metric for the test probe;
ii) determine the at least one permittivity-based metric for the reference probe; and
iii) determine the presence of the target substance and/or the concentration of the target substance based on the at least one permittivity-based metric for the test probe and the at least one permittivity-based metric for the reference probe.

20. The system of claim 19, wherein the at least one permittivity-based metric is selected from the group consisting of capacitance, dielectric constant, and impedance.

21. The system of claim 19, wherein the processor is further configured to:
determine a rate of change of the at least one permittivity-based metric for the test probe over a period of time; and
determine a rate of change of the at least one permittivity-based metric for the reference probe over the period of time.

22. The system of claim 21, wherein the processor is further configured to determine the concentration of the target substance based at least in part on:
the rate of change of the at least one permittivity-based metric for the test probe over the period of time; and
the rate of change of the at least one permittivity-based metric for the reference probe over the period of time.

23. The system of claim 19, wherein the processor is removeably coupled to the sensor.

24. The system of claim 19, further comprising a user interface coupled to the processor.

25. The system of claim 19, wherein the second test electrode is also functionalized to create a permittivity change in the area between the first and second test electrodes in the presence of the target substance.

26. The system of claim 25, wherein the second test electrode and the second reference electrode comprise a shared electrode.

27. The system of claim 26, wherein the shared second test electrode and second reference electrode are functionalized such that no permittivity change occurs in the area between the first and second reference electrodes in the presence of the target substance.

28. The system of claim 25, wherein the second reference electrode is functionalized for the target substance.

29. The system of claim 19, wherein:
the first reference electrode comprises a first reference electrode support; and
the second reference electrode comprises a second reference electrode support.

30. The system of claim 19, wherein:
the first reference electrode comprises a first reference doped region; and
the second reference electrode comprises a second reference doped region.

31. The system of claim 19, wherein:
the first test electrode comprises a first test electrode support; and
the second test electrode comprises a second test electrode support.

32. The system of claim 19, wherein:
the first test electrode comprises a first test doped region; and
the second test electrode comprises a second test doped region.

33. The system of claim 19, wherein the first test electrode and the second test electrode are functionalized with a material selected from the group consisting of glucose oxidase and oligonucleotides.

34. The system of claim 19, wherein the analyte the first test electrode and the second test electrode are functionalized for is selected from the group consisting of glucose, cholesterol, potassium, a hormone, a vitamin, a biological agent, a bacteria, a virus, a spore, mycoplasma, a prion, and a protein.

35. The system of claim 19, wherein the sensor further comprises a substrate which supports the test probe and the reference probe.

36. The system of claim 35, wherein the substrate is selected from the group consisting of silicon, glass, polymer, and quartz.

37. The system of claim 35, wherein the circuitry comprises CMOS technology.

38. The system of claim 19, wherein the second test electrode and the second reference electrode comprise a shared electrode.

39. The system of claim 19, further comprising an actuator configured to move the test probe and the reference probe from a retracted position to an engaged position.

40. The system of claim 39, wherein the actuator is manually activated.

41. The system of claim 39, wherein the actuator is coupled to the processor and activated by the processor.

42. The system of claim 39, wherein the processor is further configured to determine the at least one permittivity-based metric for the test probe and the least one permittivity-based metric for the reference probe while the test probe and the reference probe are in the engaged position.

43. The system of claim 42, wherein the processor is further configured to determine the presence and/or the concentration of the target substance while the test probe and the reference probe are in the engaged position.

44. The system of claim 19, further comprising a sensor array which comprises the sensor and at least a second sensor, wherein the at least second sensor comprises:
i) a second test probe having at least a first test electrode and a second test electrode, wherein at least the first test electrode is functionalized to create a permittivity change in the area between the first and second test electrodes in the presence of a second target substance; and
ii) a second reference probe having at least a first reference electrode.

45. The system of claim 44, wherein the target substance and the second target substance are different substances.

46. A method of determining a presence of a target substance and/or a concentration of the target substance in a fluid, comprising:
contacting a test probe having a first test electrode and a second test electrode with the fluid, wherein at least the first test electrode is functionalized to create a permittivity change in the area between the first and second test electrodes in the presence of the target substance;
contacting a reference probe having a first reference electrode and a second reference electrode with the fluid;
determining a permittivity-based metric for the test probe between the first test electrode and the second test electrode;
determining a permittivity-based metric for the reference probe between the first reference electrode and the second reference electrode; and
determining the presence of the target substance and/or the concentration of the analyte based on the test probe permittivity-based metric and the reference probe permittivity-based metric.

47. The method of claim 46, wherein the permittivity-based metric is selected from the group consisting of capacitance, impedance, and dielectric constant.

48. The method of claim 46, further comprising determining a rate of change of the permittivity-based metric over a time period.

49. The method of claim 48, further comprising displaying the rate of change of the permittivity-based metric to a user.

50. The method of claim 46, wherein the target substance is selected from the group consisting of glucose, cholesterol, potassium, a hormone, a vitamin, a biological agent, a bacteria, a virus, a spore, mycoplasma, a prion, and a protein.

51. The method of claim 46, wherein the determination of the at least one permittivity-based metric for the test probe and the least one permittivity-based metric for the reference probe occurs while the test probe and the reference probe are contacting the fluid.

52. The method of claim 51, wherein the determination of the concentration of the target substance occurs while the test probe and the reference probe are contacting the fluid.

53. The method of claim 46, further comprising displaying the concentration of the target substance to a user.

54. The method of claim 46, wherein contacting the test probe and the reference probe with the fluid comprises engaging microneedles through a subjects skin.

55. The method of claim 54, wherein the fluid comprises interstitial fluid.
